(19) United States
(12) Patent Application Publication
(10) Pub. No.: US 2007/0260167 A1
(43) Pub. Date: Nov. 8, 2007

(54) Wound protector

(76) Inventor: Janil W. Heart, Canton, GA (US)

Correspondence Address: Janil W. Heart (Ms.), 563 Mile Branch Rd, Canton, GA, 30114-7007, US

(21) Appl. No.: 10/966,458

(22) Filed: Oct. 16, 2004

(51) Int. Cl. A61F 13/00

(52) U.S. Cl. 602/60; 128/889; 602/41

(57) ABSTRACT

This invention relates to a protective spacer for protecting a wound, such a lumber spinal fusion incision, from detrimental contact or rubbing with foreign objects such as a cast surrounding or at the wound.

### BACKGROUND OF THE INVENTION

1. Field of the Invention

This invention generally relates a wound protector designed primarily to be used as a first aid product to help to protect bruises, injured areas, wounds or wounded areas, and/or incisions from contact with clothing, braces, casts or foreign objects that can disturb or rub against the scab of the wound, stitches, or treated area, possibly causing infection, re-opening, bleeding, or otherwise detrimentally effecting the treated area, and more particularly, to a wound protector that functions as a spacer between the wound and foreign objects, such as a cast, that typically contact the wound during the normal course of the patient's activities.

2. Background of the Invention.

When I was recovering from a spinal lumbar fusion, the body cast I was required to wear kept rubbing against my stitches, causing unnecessary bleeding, agitation, infection, and pain. After the stitches were removed, the cast kept opening the scab, causing more of the above-noted problem. The only product I could find to protect the wound was the BANDAID bandage which comprises a flat gauze pad mounted in the center of a plastic sheet having an adhesive backing but this product did not prevent the cast from disturbing the wound.

Subsequently, I decided to take up two small rags, rolled them into a long cylinder, and taped them inside of the cast. They were placed further apart but parallel to my incision, and held in place with duct tape. When my neurosurgeon saw this he exclaimed, “That's brilliant.” As a mother and grandmother, I remember when my children had bruises and cuts, even stitches, there was nothing to protect them from clothing, exposure, etc. other than a BANDAID-like bandage. The scabs were repeatedly opened, causing bleeding, infection, wider wounds, and larger scars as well as prolonging the time for healing. In addition BANDAID-like bandages do not allow the wound to breath freely, further increasing the time for healing.

### SUMMARY OF THE INVENTION

Accordingly, it is an object of the present invention is to provide a wound protector for providing improved healing of an injury, scab, or surgical incision which is normally subject to rubbing or contact with a foreign object, such as a cast, during the normal course of activities of the patient.

Yet another object of the present invention is to provide a wound protector that enables wounds to heal properly, without being reopened by rubbing from clothing, a cast, or any other objects.

Still another object of the present invention is to provide a wound protector that prevents contact between a wound and foreign objects, such as a cast or clothing, by forming a raised perimeter around the wound.

Another object of the present invention is to provide a wound protector that allows the wounded area to breathe, without the problems of anything irritating or disturbing the surface of the wounded area.

One advantageous feature of the invention is that it enables doctors, parents, guardians, or other caregivers to provide an extra protection between a wound and foreign objects that normally contact the wound, such as casts or clothing, by forming a raised spacer at the periphery of the wound at the skin. The results of the present invention have been wonderful and include: 1) faster healing, 2) a smaller, thinner scar and 3) fewer disruptions and/or reopening of the scab or wound

The present invention comprises a spacer element to be applied to a periphery of a wound and an attachment arrangement for attaching the spacer element at the periphery of the wound. The spacer element comprises, according to a preferred embodiment, an elongated strip having, for example, a raised cross-section. The attachment arrangement comprises, for example, an adhesive backing applied to a back of the elongated strip for holding the elongated strip at the periphery of the wound. The adhesive backing is covered by a removable layer until ready for use. The strip can be made of any size or suitable material such as deformable materials such as foam or rigid materials such as extruded plastic materials. The strip's cross-section can have different diameters and shapes, such as square or rectangular, depending upon the anticipated use of the invention.

According to a preferred embodiment of the present invention, the invention comprises:
1. A strip-like spacer;
2. A suitable attachment arrangement such as, for example, an adhesive backing applied to the spacer for attaching the space to a periphery of the wound; and
3. A bottom layer, which covers the adhesive backing until ready for use.

The strip-like spacer is to be manufactured, for example, in long strips, rolled up into coils or in long strips, which can be cut to the exact size for each need.

The primary use of this as a wound protector would be: after treating with the correct first aid procedure, parents or doctors will place the strip-like spacer of the wound protector in, for example, circles, squares or parallel lines just outside of the wounded area, but not touching the wound itself. This will form a spacer that keeps the wounded area from contacting foreign objects such as a cast or in the case of skinned knees and elbows, prevents wound from reopening or the scab from being rubbed off, displaced or inflamed by clothing and the like.

This invention is applicable to both human and animal wounds.

### BRIEF DESCRIPTION OF THE DRAWINGS

FIG. 1 is a perspective view of one embodiment of the present invention

FIG. 2 is a cross-sectional view of the embodiment of FIG. 1; and

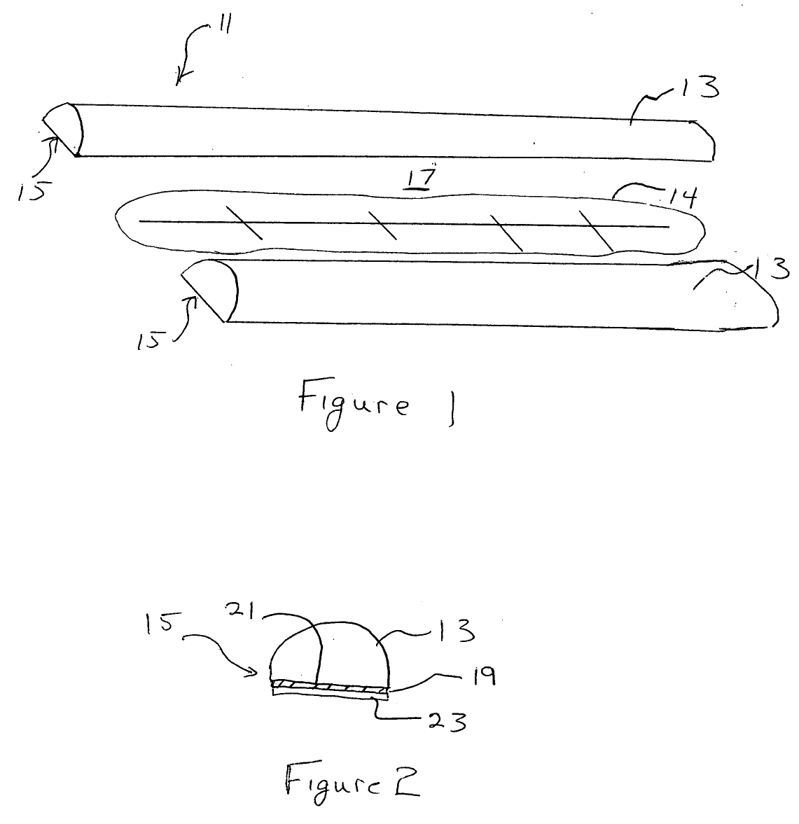

Figure is a top, planar view illustrating the use of the wound protector embodiment of FIG. 1.

### DETAILED DESCRIPTION OF THE PREFERED EMBODIMENT(S)

The wound protector of the present invention, generally shown at 11 in FIG. 1, is formed from spacers 13 to be applied at an area of the skin of the patient that is peripheral to and adjacent a wounded area 14 so that there is no contact between the spacers and wounded area. An attachment arrangement, generally shown at 15, is provided for attaching the spacers 13 to the skin 17 of the patient at the area adjacent the periphery of the wound 14. The spacers 13 have a cross-section which forms a raised perimeter around at least a portion of the periphery of the wounded area to space the wound from the foreign object and prevent incidental contact between the wounded area and the foreign object while still allowing the wounded area to breath.

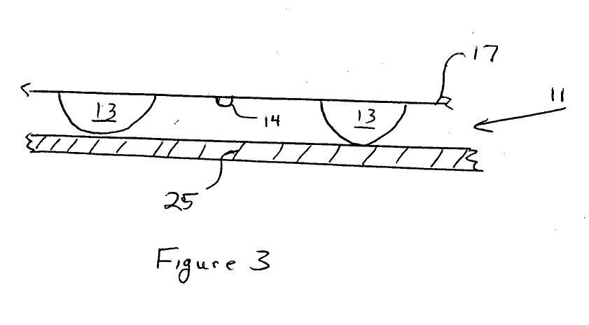

The spacer 13 comprise, according to a preferred embodiment, elongated strips having a raised cross-section. The attachment arrangement 15 comprises, for example, an adhesive backing 19, as best seen in FIG. 2, applied to a back 21 of the elongated spacer strip 13 for holding the elongated spacer strip 13 at the periphery of the wounded area 14. The adhesive backing 19 is covered by a removable layer 23 until ready for use just like a BANDAID bandage. The spacer strip 13 is made of any size or suitable elastomeric material or deformable materials such as foam. Preferable, the spacer strip 13 is made of an elastomeric material that can be extruded into elongated strips of material having a cross-section that forms a raised protuberance running substantially the length of the spacer strip 13. The spacer strip's cross-section can have different diameters and shapes, such as circular, semi-circular, elliptical, square or rectangular, depending upon the anticipated use of the invention.

The strip-like spacer 13 is to be manufactured, for example, in long strips, rolled up into coils for packaging and dispensing or packaged in long individual strips, that are then cut to the exact size for each need.

Referring to FIG. 3, the wound protector 11 is used as follows: after treating with the correct first aid procedure, parents or doctors will place the spacers 13 of the wound protector 11 in, for example, circles, squares or parallel lines just outside of the wounded area 14, but not touching the wound itself. This will form a spacer that keeps the wounded area 14 from contacting foreign objects such as a cast 25 or in the case of skinned knees and elbows, prevents the wound from reopening or the scab from being rubbed off, displaced or inflamed by clothing and the like.

This invention is applicable to both human and animal patients.

The manufactured product could be extruded with a semi-circular cross-section as shown in FIG. 1 or with any conceivable cross-sectional shape desired by the manufacturer as long as the cross-section from a protrusion running substantially along the length of the spacer 13, using any suitable material, but preferably an elastomeric material or foam material, that can be sterilized. There will be several thickness or sizes of the barrier, to accommodate smaller or larger need, and the product will be sold in rolls or packages of varying lengths.

The adhesive is selected to keep the spacer 13 in place and permit easy removed. The wound protector will then be placed in a sterilized container, opened only by the user. This container is to be placed in a box. Then the box will be placed on retail shelves, for sale.

Although the present invention has been described in terms of specific exemplary embodiments, it will be appreciated that various modifications and alterations might be made by those skilled in the art without departing from the spirit and scope of the invention as specified in the following claims.

What I claim is:

1. A wound protector for protecting a wounded area from incidental contact with a foreign object that typically contacts and disturbs the wounded area during the normal course of the patient's activities, the wound protector comprising:
a spacer having a cross-section which forms a raised perimeter around at least a portion of the periphery of the wounded area to space the wound from the foreign object and prevent incidental contact between the wounded area and the foreign object while still allowing the wounded area to breath; and
an attachment arrangement for attaching the spacer to an area of the patient which is peripheral to the wounded area.

2. A wound protector according to claim 1, wherein the spacer comprises an elongated cylinder.

3. A wound protector according to claim 1, wherein the spacer is made of an elastomeric material.

4. A wound protector according to claim 1, wherein the spacer is made of an elongated extruded elastomeric material and is rolled into a coil and cut to length as required.

5. A wound protector according to claim 1, wherein the attachment arrangement comprises adhesive to attaching the spacer directly to the patient.

6. A wound protector according to claim 1, wherein the spacer has a cross-section that forms a raised protrusion extending substantially along the length of the strip-like space

7. A wound protector according to claim 1, wherein the cross-section comprises one of a circle, a half-circle, a square, a rectangle or an ellipse.

8. A wound protector for protecting a wounded area from incidental contact with a foreign object that typically contacts and disturbs the wounded area during the normal course of the patient's activities, the wound protector comprising:
a strip-like spacer, having a cross-section that forms a raised protrusion extending substantially along the length of the strip-like spacer, for forming a raised perimeter around at least a portion of the periphery of the wounded area to space the wound from the foreign object and prevent incidental contact between the wounded area and the foreign object while still allowing the wounded area to breath;
an adhesive backing applied to the spacer for attaching the spacer at a periphery of the wound; and
a removable adhesive protective layer that covers the adhesive backing until ready for use.

9. A wound protector according to claim 8, wherein the spacer is made of an elastomeric material.

10. A wound protector according to claim 8, wherein the spacer is made of an elongated extruded elastomeric material and is rolled into a coil and cut to length as required.

11. A wound protector according to claim 8, cross-section comprises one of a circle, a half-circle, a square, a rectangle or an ellipse.
